(19) United States
(12) Patent Application Publication
(10) Pub. No.: US 2016/0278833 A1
(43) Pub. Date: Sep. 29, 2016

(54) TORQUE DRIVERS FOR HEADLESS THREADED COMPRESSION FASTENERS

(71) Applicant: WRIGHT MEDICAL TECHNOLOGY, INC., Memphis, TN (US)

(72) Inventors: Kian-Ming WONG, Lakeland, TN (US); Gary LOWERY, Eads, TN (US); Scott A. ARMACOST, Germantown, TN (US)

(73) Assignee: Wright Medical Technology, Inc., Memphis, TN (US)

(21) Appl. No.: 14/403,798

(22) PCT Filed: Oct. 6, 2014
(86) PCT No.: PCT/US14/59318

§ 371 Date: Nov. 25, 2014

(51) Int. Cl. A61B 17/86; B25B 15/00; A61B 17/88; B25B 13/08

(52) U.S. Cl. CPC A61B 17/8615; B25B 13/08; B25B 15/008; A61B 17/861; A61B 17/8665; A61B 17/8875; A61B 17/8888

(57) ABSTRACT

A threaded-shaft or fastener such as a headless bone screw, intramedullary support or the like, has a tool-receiving structure on the axial end of an externally threaded proximal end for application of torque directly to the fastener. A cannulated cap nut with internal threads complementary to the thread on the shaft can be jammed on the shaft in a tightening direction, allowing driving torque to be applied to the shaft through the cap nut for driving the fastener into a work material such as bone tissue. When the fastener has been driven to bring the cap nut up to the work material, torque is applied between tools in the fastener and cap nut, and then to the fastener alone, unjamming and advancing the fastener such that the proximal end of the fastener can be embedded in the bone tissue. The fastener is advantageously a headless bone compression screw.

### FIELD

This disclosure relates to the manipulation of fasteners such as bone screws, surgical lag bolts, intramedullary implants and the like that are externally threaded out to their proximal end and have a non-round fitting for endwise engagement by an axial tool. A cannulated jam nut is threaded over the proximal end of the fastener and engaged externally by a spanner or socket wrench. The cannulation of the jam nut also permits access to the fastener by an axial tool. The fastener is advanced into the working material, i.e., bone tissue, up to the jam nut, at least partly by using the external wrench. The fastener head is then embedded in the working material by advancing the fastener out of the jam nut and into the working material using the axial tool.

### BACKGROUND

Threaded elongated fasteners have a variety of surgical applications and a range of specific structures that are apt for different situations. Fasteners with a threaded shaft are generally termed bolts or screws but can vary in structure. Fasteners may be wholly threaded, threaded along only part of the fastener length, not threaded at all, provided with operational features such as one-way barbs, deformable expanders, receivers for intersecting other parts, or a combination of such aspects. In orthopedic applications, such fasteners may be called nails, bolts, pins, screws, beams, shafts, wires and so forth. It is necessary to consider the fastener structure and application as well as the name given to approximate the fastener's operative structures.

In threaded shaft fasteners, a thread may be provided along the entire shaft or only along part of the shaft such as the distal end. If the proximal end has a head that is wider than the fastener shaft, such as a stepwise or conical enlargement of diameter at or near the proximal end, the fastener can be threaded into working material directly or into a pilot hole or bore, until fastener head meets an obstruction such as the surface of the working material.

Due to the helical structure of the thread, turning the screw or bolt in one direction or the other advances or retracts the shaft longitudinally. The fastener can be advanced until the head abuts against the surface of the working material, or against a supporting plate, washer or other structure having an opening through which the screw shaft extends into the bone tissue, perhaps including a conical countersink or cylindrical counterbore. Additional tightening (further application of torque to advance the screw or bolt) after bringing the fastener head into contact with the obstruction exerts compression between the fastener head and the working material engaged by the thread along the more distal part of the shaft. This is useful to press and affix a structure that is under the fastener head against the working structure, or to compress distinct pieces of material together. In surgery, the screw head may compress a supporting plate against the external surface of a bone into which the fastener is threaded. The screw may pass through one segment and be threaded into another segment of a broken bone to compress displaced segments together during healing. In some arthrodesis (bone fusion) procedures, the screw may immobilize bones abutting at a joint to cause the joint to ossify and fuse.

Threads may be provided along a proximal part of a fastener shaft, either adjacent to a fastener head or on a headless fastener resembling a set screw or simple threaded shaft. In a headless fastener, the thread at the proximal end runs clear to the proximal end of the fastener shaft. A non-round axial opening can be provided in the proximal end of the shaft to receive a complementary tool for applying torque. Examples of tools are flat or Phillips screwdrivers, hexagonal Allen wrenches, variants with star or spline shaped non-round axial openings, etc. An axial fastener can also comprise a spanner type nut, which is recessed in the axial end for receiving a socket.

Both proximal and distal lengths along a fastener may be threaded, and the threads can be of different character. In certain hanger bolts, for example, the distal length is a lag screw with tapered point and wood-engaging distal threads. The proximal end is a stub that is threaded like a machine screw to receive a nut. However the proximal end is not structured or intended to be embedded in the wood.

Bone compression fasteners may advantageously be distinctly threaded along different portions. A head may be spaced by a smooth unthreaded shaft from a distal thread, for exerting a longitudinal force. It is an aspect of bone fasteners that the bone tissue to be traversed by a fastener may include distinct zones of dense load bearing cortical bone tissue, particularly near a surface of the bone, less dense or porous cancellous bone tissue in the internal part of the bone, and a central medullary opening. Different thread structures may be optimal for the different tissues.

In a bone compression screw, proximal and distal thread portions can have different diameters and also different thread pitches (namely different ratios for the length of axial advance versus unit of rotation). When spaced threaded portions of different thread pitches are engaged in bone tissue at axially spaced locations, rotation of the fastener exerts longitudinal force along the fastener shaft in one direction or the other.

In an advantageous “headless” compression fastener known as a Herbert screw, for example, a proximal threaded length has a diameter larger than the diameter of a smooth fastener shaft leading to distal threaded length, and the distal thread has a longer pitch than the proximal thread. When the fastener is driven into bone tissue (rotated in a bore or in a self-tapping manner), the distal thread engages bone tissue spaced from the surface and advances the fastener longitudinally, at a rate (longitudinal advance per unit of rotation) determined by the distal thread pitch. When the more proximal thread comes and engages the bone surface, continued driving causes the proximal end to become embedded in the bone tissue. However, because the proximal thread has a smaller thread pitch (less axial advance per unit of rotation) than the distal thread, driving the fastener to embed the proximal threaded end in the bone also applies increasing compression between the proximal and distal ends of the fastener. This aspect is useful, for example, to draw together and heal broken bone sections or to abut and immobilize bone sections that are to be fused.

It is possible to envision the pitch at the proximal end being longer than the pitch at the distal end. If so, when the proximal and distal ends are embedded in bone tissue at axially spaced points along the fastener, continued threading advances the proximal end into the tissue at a rate that is greater than the advance of the distal end, applying tension to push the proximal and distal sections apart.

A threaded fastener with distinct threads might or might not have a difference in diameter. However an enlarged screw or bolt head is advantageous to provide a fresh inside diameter for a wider threaded part to engage after passage of a narrower shaft.

For simplicity, all threaded compression fasteners for bones will be termed bone “screws” in this disclosure, it being understood that the term “screw” does not require a screw head or any particular configuration of threads unless expressly stated or apparent from the context. Likewise, the term “screw” should be deemed to apply to various configurations of shafts and heads, having at least some thread related functional aspects.

In particular bone compression fasteners, a proximal end of a shaft can be advanced until the proximal end the fastener is at least flush with a defined tissue surface or may be embedded below the tissue surface. This structure and function are particularly useful for fixing the proximal end of the fastener in dense cortical bone tissue at the surface of a bone. The dense cortical tissue provides a robust base that supports the embedded fastener.

Because the proximal thread runs to the proximal end of the screw shaft, such a screw may be considered “headless,” although such a fastener might be characterized by an increased diameter at the proximal end that could be termed an externally threaded head. The pitch of the threads of a headless fastener likewise might or might not differ from the pitch along the shaft. Because the outside surface of the proximal end of the fastener is occupied by threads, torque is applied by engaging a non-round shape at the axial end of the fastener with a tool having a complementary shape. A non-round faceted or splined axial opening receives a complementary driver such as a screwdriver (flat or Phillips), a hexagonal Allen wrench, a splined star driver, Torx driver, a socket that fits over a non-round hexagonal or similar axial protrusion on the fastener, or a similar torque transfer coupling.

The non-round structures for engagement of the tool and the fastener to transmit torque can be male/female or female/male, or a combination of the two. But the tool-to-fastener engagement structures are (or include) non-round shapes that are smaller than the outside diameter of the proximal end of the fastener. The diameter of the engageable torque transmitting structures limits the force that can be applied without stripping the non-round structures on the fastener or on the tool.

Turning force (torque) is a matter of force times radius. A given degree of torque can be applied by a smaller force at a greater lever arm radius or by a greater force at a shorter lever arm radius. Conversely, when applying a given degree of torque to a fastener, non-round tool engaging structures of a small radius experience more material strass then non-round structures of larger radius transmitting the same degree of torque. It is not difficult inadvertently to mar or strip the torque coupling driving structures of a fastener or a tool by applying overly vigorous torque or by failing to accurately align and fully insert the driver tool into the complementary opening in the proximal end of the fastener before applying torque.

It would be advantageous to develop a way to apply torque to fastener by engaging over a larger radial span than is possible using the surfaces of an axially inserted tool that may be vulnerable to stripping. Hexagonal outer spanner surfaces such as commonly provided on bolt heads have a larger radius, but a bolt head is precluded. The outside thread of the fastener head cannot be engaged at the outside diameter of the fastener head because the outside diameter is occupied by the thread and the thread is necessary to embed the headless fastener.

### SUMMARY

An object of this disclosure is to improve the usefulness and convenience of fasteners that are threaded up to the proximal end of a fastener shaft and have a non-round tool receiving structure that requires an axially inserted driving tool such as an Allen wrench, splined or star-shaped wrench, axial socket fixture or the like for application of driving torque. In particular, an object is to facilitate the embedment of headless compression screws and the like into bone tissue.

These and other objects are met by a headless threaded-shaft or externally-threaded-head type fastener, having axial torque tool receiving surfaces in the proximal end of the fastener, in combination with a cannulated cap nut that has an internal thread complementary to the external thread on the shaft or head of the fastener.

The cap nut has an axial blocking flange or another form of thread disruption at a proximal point along the internal thread below which the distal part of the internal thread can be threaded onto the fastener. Thus the cap nut can be threaded onto the fastener up to a point at which the proximal (rear) end of the fastener jams axially against the blocking flange or encounters the thread disruption and jams. At this point, torque may be applied to the cap nut in the same direction as needed to advance the threaded fastener into the bone tissue. The torque is coupled through the cap nut to turn the fastener.

Torque on the cap nut can be applied, for example, against tool-receiving non-round surfaces such external hexagonal bolt-like flats for engagement by a spanner wrench, socket wrench or similar tool that engages against the outside non-round surfaces of the cap nut. These external surfaces of the cap nut are at radial distance that exceeds the size of the fastener head, and can exceed by several times the diameter of axial tool engaging structures at the end of the fastener. Alternatively, a hex opening or splines or socket receiver or other non-round engagement structure can be provided on the proximal side of the cap nut, but having a span that is larger than the span of the tool receiving opening in the fastener. Tool engaging structures on the axial end of the cap nut accommodate a cannulation (i.e., an axial hole), arranged and sized so as to admit the axial wrench or other tool through the cap nut to engage the tool receptacle at the axial end of the fastener.

The distal thread and the proximal head thread on the fastener (e.g., compression screw) are pitched in the same direction (both are right handed or both are left handed. Accordingly, the cap nut can be threaded onto the proximal thread and advanced to the point that the fastener is jammed in the cap nut, which can be threaded no further due to the fastener head encountering an obstruction such as an annular flange. At this point, torque applied to the cap nut in the tightening direction is coupled to the fastener, which is rigidly attached to the cap nut with respect to torque applied in the tightening direction. Torque is applied to the cap nut to advance the distally threaded fastener shaft into the tissue.

When advance of the fastener brings the cap nut to the tissue surface, the cap nut is held stationary, e.g., with a spanner wrench. The fastener is torqued to advance further in the tightening direction using a torque tool applied to the axial end of the fastener which holding the cap nut. Tightening then causes the fastener to be unjammed and threaded out of the cap nut and toward the tissue. Once the fastener is unjammed, cap nut can be threaded rearwardly in the loosening direction and removed. Advantageously, the fastener is advanced such that the externally threaded proximal head is embedded in the tissue below the surface.

The invention extends to apparatus and method aspects as described, and is particularly apt for relatively small compression fasteners and for fasteners that are used for arthrodesis and arthroplasty surgical procedures involving the bones of the mid-foot, forefoot, ankle, hand, wrist and the like.

### BRIEF DESCRIPTION OF THE DRAWINGS

These and other objects and aspects will be appreciated by the following discussion of preferred embodiments and examples, with reference to the accompanying drawings, and wherein:

FIGS. 1a-1c and 2a-2b illustrate a cannulated cap nut configuration and a compression screw with an internally threaded head, respectively, arranged for manipulation in conjunction with one another as shown in FIGS. 3 and 4.

FIGS. 1a, 1b, 1c are top plan, side elevation (sectional) and bottom plan views of the cap nut.

FIGS. 2a, 2b are top plan and side elevation views of the compression screw.

FIG. 3 is a side elevation, partly in section, illustrating manipulation of the compression screw using the cannulated cap nut together with spanner and axial wrenches.

FIG. 4 is a side elevation, partly in section, showing the compression screw in final position in bone tissue.

### DETAILED DESCRIPTION OF EXEMPLARY EMBODIMENTS

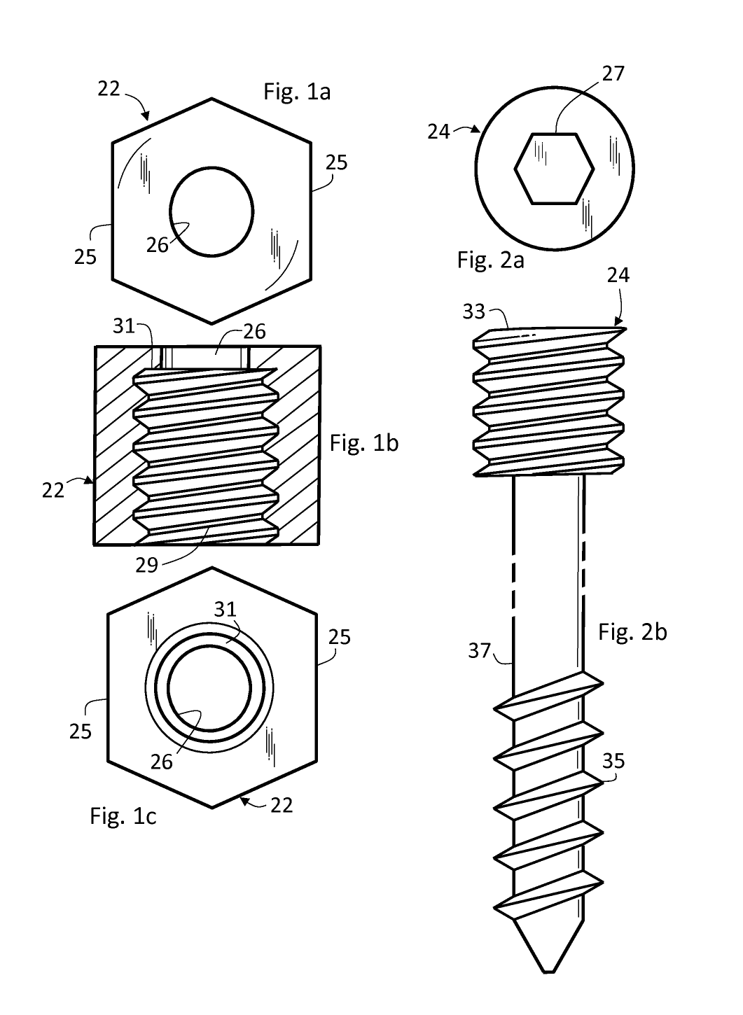

A cap nut 22 as shown in FIGS. 1a-1c is internally threaded to complement and to receive temporarily the externally threaded proximal end of a “headless” compression screw 24. More particularly, the cap nut 22 and compression screw 24 are threaded together until the cap nut 22 and the compression screw 24 are rigidly fixed together against rotation relative to one another. The non-round shape of the cap nut 22 is used as the point of application of a first tool. When the cap nut 22 and the compression screw are rigidly fixed to one another, application of the first tool to apply torque to the cap nut 22 also applies torque to compression screw 24 fixed in the cap nut 22. The compression screw 24 can thereby be threaded into a work material such as bone tissue in a self-tapping manner or into a prepared pilot hole in the work material.

The cap nut 22 can be rotated relative to the compression screw 24, either to thread the cap nut 22 and compression screw 24 together while they are freely rotatable and threadable longitudinally toward one another, or to separate the compression screw 24 from the cap nut 22 by threading them longitudinally apart. Threading the cap nut 22 and compression screw longitudinally or axially toward and apart from on another involves relative rotation of the cap nut 22 and compression screw 24 in one rotational direction or other. However it does not matter whether either one of nut 22 or screw 24 is held stationary while the other is rotated, or if both are rotated in opposite directions.

When threading the cap nut 22 and the compression screw 24 toward one another, the cap nut 22 and the compression screw 24 become fixed because their structures cause the compression screw 24 to jam in the cap nut 22. In the embodiment shown in FIGS. 1a-1c, an obstruction in the form of an inner flange 31 is provided inside the cap nut. The internal flange as an inside diameter that is less than the diameter of the female threads 29 in the cap nut 22.

Threading together the cap nut 22 and compression screw 24 can proceed by applying torque between cap nut 22 and compression screw 24. Torque in the tightening direction causes the proximal end 33 of the compression screw (FIG. 2b) to advance axially in the cap nut 22. Eventually, the end 33 of the compression screw 24 jams against the inner flange 31 of the cap nut 22. Once the nut 22 and screw 24 jam together in this way, application of torque to one of nut 22 and screw 24, at least in the direction that continues to jam them together, couples that torque also to the other of nut 22 and screw 24. In this way application of torque to the cap nut 22 can be coupled through the “headless” threaded proximal end of compression screw 24 to advance the distal thread 35 along the shaft 37 of compression screw 24, into a workpiece such as a bone or segment of bone.

Application of torque in the opposite direction, namely the rotational direction that would thread the cap nut 22 and the compression screw apart, can separate the jammed nut 22 and screw 24. Enough torque is needed first to unjam the nut 22 and screw 24, which may require two tools to engage the nut 22 and screw 24 to apply a torque or relative rotation force between them. Once unjammed, the nut 22 can be held externally (optionally using a first tool for holding nut 22) while applying torque to the screw 24, e.g., using an axially inserted non-round second tool complementary to the fitting 27 in the end 33 of the compression screw 24. The tool is inserted through the cannulation or hole 26 in the cap nut 22.

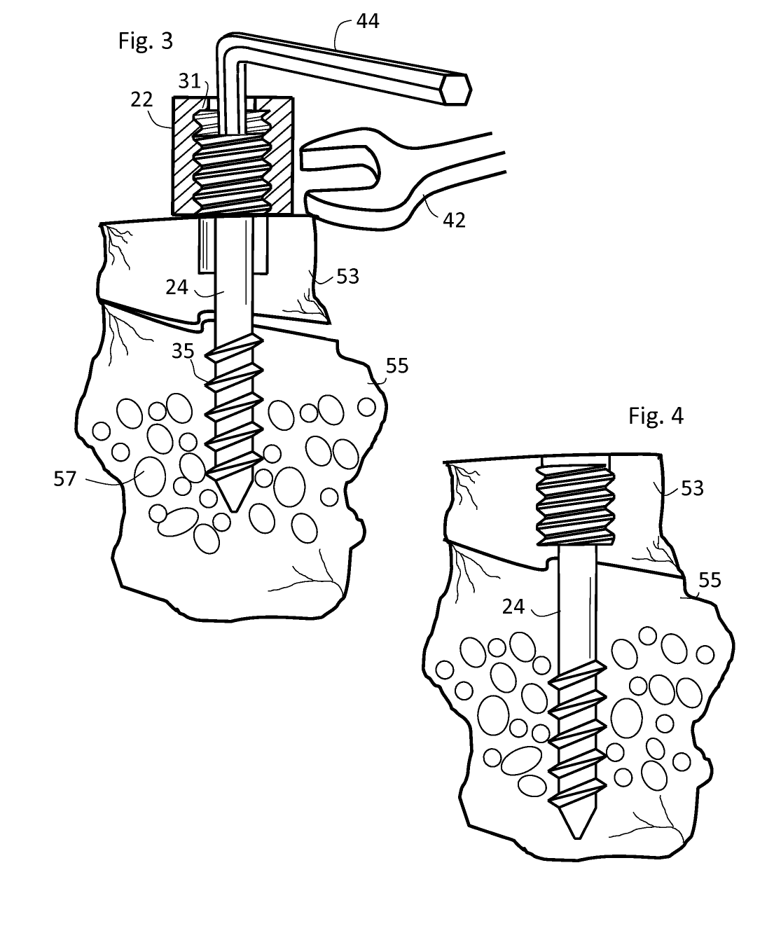

FIGS. 3 and 4 demonstrate one possible application of the structures described, namely to set a compression screw 24 in place to apply tension between bone segments 53, 55. In FIG. 3, the cap nut 22 is shown in section, threaded onto the proximal threaded end of compression screw 24. More particularly, cap nut 22 and compression screw are jammed together by applying torque between nut 22 and screw 24 using tools 42, 44. In this embodiment, the first tool is a spanner wrench 42 and the second tool is an Allen wrench 44. Torque can be applied to advance the distal thread 35 of the compression screw 24 into bone segment 55 by application of torque to the compression screw 24 via the cap nut 22 jammed thereon, namely by applying torque to the cap nut 22 using spanner wrench 22. The rotational direction of torque applied to cap nut 22 to advance thread 35 into bone segment 55 (e.g., a cancellous area 57) is the same direction that tightens the cap nut 22 onto the proximal end of the compression screw 24. This keeps the compression screw jammed against the inner flange 31 in the cap nut 22.

In the state shown in FIG. 3, the assembly of the cap nut 22 and compression screw 37 have been axially advanced until the cap nut abuts against the surface of bone segment 53. At that point, the cap nut 22 is held stationary using spanner wrench 42 and the compression screw 24 is unjammed and threaded forward and out from cap nut 22, using the Allen wrench applied through the cannulation hole in cap nut 42.

There are various configurations of compression screw threads and shafts. In order to clearly demonstrate the subject matter, the threaded proximal end of compression screw 24 is shown with a considerably larger diameter than the shaft or compression screw 24. In such an embodiment, a counterbore may be provided in bone segment 53 (e.g., a dense cortical area of the bone segment 53) to receive the compression screw. The compression screw 24 can be advanced axially using tool 44 and/or the cap nut can be retracted and removed from the compression screw 24, to facilitate advancing the compression screw to the final position shown in FIG. 4. In the depicted embodiment, the pitch of the threads along the proximal head and distal shaft differ, with the distal thread pitch being at least slightly greater than the proximal head thread pitch. As the compression screw is threaded into its final position, for a given rotational advance, the distal thread advances axially further in segment 55 than the proximal thread advances into segment 53. This pulls segments 53, 55 together into abutment.

The cap nut 22 has an external non-round shape configured to receive a tool as discussed below. In the depicted embodiment, the non-round shape of the cap nut 22 is defined, for example, by opposite parallel flat sides or faces 25 that can receive a spanner wrench or other similar wrench for application of torque to the cap nut 22. The depicted cap nut 22 is hexagonal. In other embodiments, the cap nut can have a different number of faces 25, e.g., defining a square or other polygonal shape. Alternatively, the cap nut 22 may be splined.

It should be appreciated that the shapes of the cap nut compression screw and tools are subject to variations in their types and genders, and still can be capable of access to the cap nut or compression screw, respectively and for application of torque. Instead of a spanner wrench, a tool for the cap nut could comprise a socket or pliers, for example. The end 33 of the compression screw was is not required to have a female hex opening and could have a different shape or even a male non-round shape to be engaged by a nut driver socket. These and similar variations are possible means for application of torque.

Accordingly, the subject matter shown and described involves a combination of a threaded screw such as a compression screw 24 or similar surgical fastener, a cannulated cap nut 22 threadable on the screw 24, and at least one tool 44 for applying driving torque. The compression screw 24 or other surgical fastener comprises a shaft 37 to be embedded in tissue 53, 55 at least along an axial part such an externally threaded proximal end of the fastener 24. The extreme proximal end 33 has an axially facing non-round fitting 27 for receiving a fastener wrench 44 for applying driving torque directly to the compression screw 24 or other surgical fastener.

The cannulated cap nut 22 is configured for application of torque to the cap nut 22, for example using a wrench 42 to hold and/or turn cap nut 22. The cap nut 22 has an axial opening 26 sized to admit the fastener wrench 44. The cap nut 22 is internally threaded along a distal axial distance limited proximally by a thread stoppage such as inner flange 31 or by a similar obstruction. The cap nut 22 threadably engages the proximal end of the compression screw or other surgical fastener 24 up to the thread stoppage such as flange 31.

Threading the screw or fastener 24 into the cap nut 22 in a tightening direction up to the thread stoppage 31 enables application of torque to the screw or fastener 24 via application of torque to the cap nut 22. Application of torque to the screw or fastener 24 in a loosening direction relative to the cap nut 22, via the fastener wrench 44 in the axially facing fitting while holding the cap nut 22, enables the screw or fastener 24 to advance beyond from the cap nut 22. In a compression screw embodiment, robust torque can be applied to the assembly of the compression screw 24 and cap nut 22 via a tool 42 applied to the cap nut 22, to thread the fastener into bone tissue. When the assembly has been advanced to bring the cap nut up to the bone tissue, first and second tools 42, 44 are used to apply torque in a loosening direction to unjam the assembly of the cap nut 22 and compression screw 24. The cap nut 22 can be held or threaded backward off the threaded proximal head of the compression screw 24, and the compression screw 24 can be threaded using the second tool 44 engaged in the compression screw to thread the proximal head of the compression screw into the bone tissue.

In the nonlimiting embodiments discussed as examples, the cap nut 22 comprises non-round surfaces 25 such as wrench flats for receiving a wrench for application of torque to the cap nut. The cap nut 22 can comprises external hexagonal flat surfaces for receiving a spanner wrench.

The surgical fastener can be an intramedullary fastener, bone screw or device for application of tension or compression. The fastener has a proximal threaded end on a shaft leading to a distal grasping structure. The grasping structure can include a threaded distal portion 35 to be driven into the tissue. In a compression screw embodiment, the threaded distal portion and the threaded proximal end can have different thread pitches, whereby force is exerted between the distal portion and the proximal end, in particular tension to apply force between and potentially to draw together tissues that are respectively engaged by the proximal and distal threaded portions. In that case, the threaded distal portion has a longer thread pitch than the proximal end whereby compression force is exerted between the distal portion and the proximal end.

Whether the tools and their engaged surfaces are male/female or female/male, the span of the engaged and engaging surfaces of the cap nut 22 and its associated tool are readily made considerably wider than the axial opening 26 in the cannulated cap nut 22 or the tool receiving end 33 of the compression screw or similar fastener. The cap nut 22 has an internal thread complementary with the external thread on the proximal end or head of the “headless” fastener. The cap nut 22 can be threaded down onto the screw 24 to jam against an annular flange 31 or other obstruction in the cap nut 22. In an alternative embodiment, two distinct cap nuts can be threaded on the proximal end of the screw and jammed together to provide a temporarily attached structure for applying torque to the screw. Torque applied to the jammed cap nut set of cap nuts in the tightening direction rotates the fastener and drives the distal thread 35 forward into the bone tissue 53, 55.

When the cap nut 22 is advanced up to the surface of the bone tissue, a wrench such as an Allen, star, spline, Torx, nut driver or the like, small enough to pass through the cannulation in the cap nut, is inserted to engage directly in a complementary wrench receiving fitting 27 in the proximal end 33 of the screw or fastener 24. While holding the cap nut 22, e.g., with a spanner wrench or the like, the externally threaded proximal end of the fastener 24 is then threaded out of its jammed position in the cap nut 22 and into the bone tissue. The now-loosened (un-jammed) cap nut can be loosened back off the fastener and/or the fastener can be advanced by turning the fastener while holding the cap nut. The fastener can be threaded into the bone tissue in a self-tapping manner or with a reamed bore as shown in FIG. 3, or a drilled pilot hole or the like.

The cap nut axially covers the threaded proximal end of a “headless” threaded fastener such as a compression screw, and serves to radially enlarge the dimensions of the structure that can be engaged with a tool to apply torque. The headless threaded part of the screw 24 has an externally threaded enlargement that has a greater diameter than the diameter of the more distal fastener shaft 37. When the fastener or screw 24 has been advanced to the point at which the distal side of the cap nut 22 abuts the tissue, the distal part of the threaded enlargement is in position to commence threading into the tissue.

The proximal thread of the fastener need not reside entirely within the cap nut 22 in the jammed position and can protrude axially so that the proximal thread, for example of a “headless” compression screw, can be started into the bone tissue by application of torque to the cap nut 22. When the jam nut abuts the bone tissue, however, the jam nut 22 can be held against rotation using one tool 42. The axially inserted second tool 44, such as an Allen wrench, splined or star-shaped wrench, Phillips or flat screwdriver, or similar driving tool is inserted through the cannulated axial opening in the jam nut. The fastener-driving tool engages with the non-round tool receiving structure in the fastener, which is typically a female shape. Torque is applied directly to the fastener 24 which holding the cap nut 22. The fastener is thereby threaded away from the thread obstruction and the proximal end of the externally threaded fastener advances into the bone tissue.

Advantageously, the fastener is a compression screw, bolt or the like wherein a distal part of the fastener has a thread with a longer thread pitch and the proximal headless end of the fastener has a thread with a shorter thread pitch. Thus, with rotation of the fastener, compression is applied to an increasing extent between the proximal and distal threaded parts.

In addition to a combined fastener such as a bone compression screw with an externally threaded proximal end on a shaft, and a tool receiving structure on axial end of the proximal end of the shaft, and a cannulated cap nut having an external structure configured to receive a torque applying tool, an internal thread complementary to the proximal end of the shaft and a cannulated jamming cap nut, this disclosure entails a method for setting a bone compression screw. The method as described includes threading the cap nut onto the shaft in a relative rotation direction of the threaded end and the internal thread sufficiently to jam the fastener in the cap nut. The fastener is threaded into the workpiece, such as bone tissue, by applying torque to the cap nut in the relative rotation direction, thereby maintaining a jammed condition of the cap nut and the fastener while advancing the fastener into the workpiece. At least one of the cap nut and the fastener is engaged with a tool and torque is applied in a loosening direction to unjam the fastener from the cap nut. This may concern holding one of the fastener and the cap nut while applying torque to the other, or relatively moving both the fastener and the cap nut. Continued torque is then applied, at least to the fastener, to advance the fastener into the bone tissue or other workpiece. This continued torque is applied using a tool applied to the tool receiving structure on the axial end of the proximal end of the shaft.

Threading the fastener into the bone tissue or other workpiece by applying torque to the cap nut in the relative rotation direction as described can include advancing the fastener to bring the cap nut up to a surface of the bone tissue before applying said torque in the loosening direction. The method can be completed by fully embedding the externally threaded proximal end of the shaft in the bone tissue.

The invention has been disclosed in connection with certain embodiments having attributes that are advantageous for the reasons described. These attributes can be realized together or individually and with other features without departing from the invention. Reference should be made to the appended claims as opposed to the foregoing description of embodiments and examples, in order to assess the scope of the invention claimed.

What is claimed is:

1. In combination, a threaded surgical fastener, a cannulated cap nut and at least one tool for applying driving torque,
wherein the surgical fastener comprises a shaft to be embedded in tissue at least along an axial part of an externally threaded proximal end of the surgical fastener, and the proximal end has an axially facing non-round fitting for receiving a fastener wrench for applying said driving torque directly to the surgical fastener;
wherein the cannulated cap nut is configured for application of torque to the cap nut, and an axial opening sized to admit the fastener wrench, the cap nut being internally threaded along a distal axial distance limited proximally by a thread stoppage, such that the cap nut threadably engages the proximal end of the surgical fastener up to the thread stoppage,
whereby threading the fastener into the cap nut up to the thread stoppage enables application of torque to the fastener via application of torque to the cap nut, and application of torque to the fastener relative to the cap nut, via the fastener wrench in the axially facing fitting, enables the fastener to advance from the cap nut into the tissue.

2. The combination of claim 1, wherein the cap nut comprises non-round surfaces for receiving a wrench for application of torque to the cap nut.

3. The combination of claim 1, wherein the cap nut comprises external hexagonal flat surfaces for receiving a spanner wrench.

4. The combination of claim 1, wherein the surgical fastener comprises a shaft with a threaded distal portion to be driven into the tissue, and wherein the threaded distal portion and the threaded proximal end have different thread pitches, whereby force is exerted between the distal portion and the proximal end.

5. The combination of claim 4, wherein the threaded distal portion has a longer thread pitch than the proximal end whereby compression force is exerted between the distal portion and the proximal end.

6. A driving arrangement, comprising:
a compression bone screw having an externally threaded proximal end on a shaft, and a tool receiving structure on axial end of the proximal end of the shaft;
a cannulated cap nut having an external structure configured to receive a torque applying tool, an internal thread complementary to the proximal end of the shaft, a thread obstruction defining a jamming structure when threaded onto the shaft, and an axial opening permitting access to the tool receiving structure on the axial end of the proximal end of the shaft.

7. The driving arrangement of claim 6, wherein the cannulated cap nut comprises a hex nut with an inner flange for engaging the compression bone screw when threaded onto the proximal end of the shaft.

8. The driving arrangement of claim 7, wherein the tool receiving structure on the proximal end of the shaft comprises a non-round female structure for receiving the torque applying tool.

9. The driving arrangement of claim 8, wherein externally threaded proximal of the shaft extends beyond the cap nut for commencing engagement in a work material.

10. A method for setting a bone compression screw, comprising:
providing a fastener with an externally threaded proximal end on a shaft, and a tool receiving structure on axial end of the proximal end of the shaft; and a cannulated cap nut having an external structure configured to receive a torque applying tool, an internal thread complementary to the proximal end of the shaft, a thread obstruction defining a jamming structure when threaded onto the shaft, and an axial opening permitting access to the tool receiving structure on the axial end of the proximal end of the shaft;
threading the cap nut onto the shaft in a relative rotation direction of the threaded end and the internal thread sufficiently to jam the fastener in the cap nut;
threading the fastener into a workpiece by applying torque to the cap nut in said relative rotation direction, thereby maintaining a jammed condition of the cap nut and the fastener while advancing the fastener into the workpiece;
engaging at least one of the cap nut and the fastener with a tool and applying torque in a loosening direction to unjam the fastener from the cap nut;
applying continued torque to advance the fastener into the workpiece, using a tool applied to the tool receiving structure on the axial end of the proximal end of the shaft.

11. The method of claim 10, wherein threading the fastener into the workpiece by applying torque to the cap nut in said relative rotation direction includes advancing the fastener to bring the cap nut up to a surface of the workpiece before applying said torque in the loosening direction.

12. The method of claim 11, comprising embedding the externally threaded proximal end of the shaft in the workpiece.
